(19) United States
(12) Patent Application Publication
(10) Pub. No.: US 2024/0099563 A1
(43) Pub. Date: Mar. 28, 2024

(54) MANIFOLD DEVICES, ASSEMBLIES, AND METHODS FOR ENDOSCOPE SYSTEMS

(71) Applicant: Boston Scientific Scimed, Inc., Maple Grove, MN (US)

(72) Inventors: Ryan V. Wales, Northborough, MA (US); Paul Smith, Smithfield, RI (US); John B. Golden, Norton, MA (US); Scott E. Brechbiel, Acton, MA (US); Jeff Gray, Sudbury, MA (US); Kurt Nicholas Robakiewicz, Upton, MA (US)

(73) Assignee: Boston Scientific Scimed, Inc., Maple Grove, MN (US)

(21) Appl. No.: 18/470,225

(22) Filed: Sep. 19, 2023

Related U.S. Application Data
(60) Provisional application No. 63/410,173, filed on Sep. 26, 2022.

(51) Int. Cl. A61B 1/00; A61B 1/015; A61B 1/12

(52) U.S. Cl. CPC A61B 1/00128; A61B 1/00119; A61B 1/015; A61B 1/126

(57) ABSTRACT

Devices, systems, and methods for coupling tubing to a fluid reservoir to provide liquid to an endoscope system. A manifold for coupling to a fluid reservoir and a tubing assembly may include first portion interfacing with an interior of the fluid reservoir and a second portion configured to interface with tubing to be fluidly coupled to the interior of the reservoir. The first portion may include one or more openings to allow gas to pass into the fluid reservoir and liquid to pass out of the fluid reservoir. The second portion may include one or more ports for connecting to tubing that brings gas to the fluid reservoir and receives liquid from the fluid reservoir. The manifold may include a valve that selectively allows gas to pass into the fluid reservoir.

### CROSS-REFERENCE TO RELATED APPLICATIONS

This application claims the benefit of U.S. Provisional Patent Application Ser. No. 63/410,173 filed on Sep. 26, 2022, the disclosure of which is incorporated herein by reference.

### TECHNICAL FIELD

This disclosure relates generally to manifold devices, assemblies and methods, and particularly to manifold devices, assemblies, and methods for an endoscope system.

### BACKGROUND

A wide variety of intracorporeal and extracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a manifold configured to couple to a fluid source and a tubing assembly in fluid communication with an endoscope may include a first portion configured to be internal to the fluid source, the first portion comprising a first opening in fluid communication with a first through hole and a second opening in fluid communication with a second through hole, and a second portion configured to be external to the fluid source, the second portion comprising a first port in fluid communication with the first through hole, the first port is configured to couple with a first tubing configured to be in fluid communication with the endoscope and a second port in fluid communication with the second through hole, the second port is configured to couple to a second tubing to be in fluid communication with the endoscope.

Alternatively or additionally to any of the examples above, in another example, the manifold may include a third opening in the first portion, the third opening is in fluid communication with a third through hole and the first port, and a valve in fluid communication with the third opening, wherein when pressure in the third through hole reaches or goes beyond a threshold, the valve allows gas to output from the third opening, and when pressure in the third through hole has not reached the threshold, the valve prevents gas from exiting through the third opening.

Alternatively or additionally to any of the examples above, in another example, the first portion may comprise a third opening in fluid communication with a third through hole and the first port, the second portion may comprise a third port in fluid communication with the third through hole and the third opening, and the third opening is configured to receive gas passing through one or both of the first port and the third port.

Alternatively or additionally to any of the examples above, in another example, the manifold may include an interface configured to engage a wall of the fluid source and seal an opening in the wall of the fluid source in which one or both of the first portion and the second portion extends.

Alternatively or additionally to any of the examples above, in another example, the interface may define a perimeter having a portion that is configured to be parallel to a bottom surface of the fluid source.

Alternatively or additionally to any of the examples above, in another example, the manifold may include a connector configured to connect the first portion to the second portion.

Alternatively or additionally to any of the examples above, in another example, the connector may include a first set of threads extending around the first portion, a second set of threads extending around the second portion, and the first set of threads and the second set of threads are configured to threadedly engage one another to secure the first portion and the second portion to the fluid source.

Alternatively or additionally to any of the examples above, in another example, the manifold may include an actuator configured to be adjusted to adjust flow of fluid through the second portion.

Alternatively or additionally to any of the examples above, in another example, when the actuator is in a first position, liquid in the fluid source may be allowed to flow from the fluid source through the first port, and when the actuator is in a second position, liquid in the fluid source may be prevented from flowing from the fluid source through the first port.

Alternatively or additionally to any of the examples above, in another example, the actuator may be configured to couple the first portion with the second portion.

In another example, a fluid reservoir assembly configured to couple to a tubing assembly in fluid communication with an endoscope, the fluid reservoir assembly may comprise a container having a first opening and a second opening, a cap configured to couple to the container and cover the first opening, and a manifold configured to couple to the container and cover the second opening, and the manifold having a first port and a second port positioned exterior of the container when the manifold is coupled to the container, wherein the first port and the second port may be in fluid communication with an interior of the container.

Alternatively or additionally to any of the examples above, in another example, the manifold may include a third port, wherein when the manifold is coupled to the container, third port may be positioned exterior of the container and in fluid communication with the interior of the container.

Alternatively or additionally to any of the examples above, in another example, the container has a first end, a second end, and a side wall extending between the first end and the second and, and the first opening extends through the first end and the second opening extends through the side wall proximate the second end.

Alternatively or additionally to any of the examples above, in another example, the manifold may comprise a perimeter, a portion of which is parallel to the second end of the container.

Alternatively or additionally to any of the examples above, in another example, the manifold comprises a first portion configured to be positioned exterior of the container and a second portion configured to be positioned in the interior of the container, and the first portion and the second portion are configured to couple to one another and fluidly seal the first opening.

In a further example, a medical device assembly comprises an endoscope, a tubing assembly configured to couple to the endoscope, the tubing assembly having a first tubing, a fluid reservoir, and a manifold, the manifold is configured to couple to the fluid reservoir, the may manifold include a first port configured to be in fluid communication with liquid in the fluid reservoir and the first tubing of the tubing assembly, and a second port configured to be in fluid communication with a pressurized gas, an interior of the fluid reservoir, and the first tubing of the tubing assembly.

Alternatively or additionally to any of the examples above, in another example, the manifold may include a third port configured to be in fluid communication with the liquid in the fluid reservoir and a second tubing of the tubing assembly.

Alternatively or additionally to any of the examples above, in another example, the fluid reservoir has a first end, a second end, a side wall extending between the first end and the second end, and an opening extending through the side wall, and the manifold is configured to extend through the opening in the side wall.

Alternatively or additionally to any of the examples above, in another example, the manifold may comprise a first portion configured to be positioned exterior of the fluid reservoir and a second portion configured to be positioned in the interior of the fluid reservoir, and the first portion and the second portion are configured to couple to one another and fluidly seal the opening.

Alternatively or additionally to any of the examples above, in another example, the manifold may include a one-way valve configured to be positioned interior of the fluid reservoir.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3A depicts a schematic view of an illustrative endoscope system, wherein the endoscope system is activated to deliver air to atmosphere;

FIG. 3B depicts a schematic view of an illustrative endoscope system, wherein the endoscope system is activated to deliver air to a patient through the patient end of the endoscope;

FIG. 3C depicts a schematic view of an illustrative endoscope system, wherein the endoscope system is activated to deliver lens wash fluid through the patient end of the endoscope;

FIG. 3D depicts a schematic view of an illustrative endoscope system, wherein the endoscope system is activated to deliver irrigation fluid through the patient end of the endoscope;

FIG. 4 depicts a schematic view of an illustrative manifold coupled to a fluid source and tubing assembly of an endoscope system, with the manifold and tubing assembly depicted in cross-section;

FIG. 5 depicts a schematic cross-section view of an illustrative manifold;

FIG. 6 depicts a schematic cross-section view of an illustrative manifold coupled to tubing and a side wall of a fluid source;

FIGS. 7A and 7B depict a schematic cross-section views of an illustrative manifold with a valve;

FIG. 8 depicts a schematic perspective view of an illustrative manifold;

FIG. 9 depicts a schematic perspective view of a first portion of the illustrative manifold depicted in FIG. 8;

FIG. 10 depicts a schematic perspective view of a second portion of the illustrative manifold depicted in FIG. 8;

FIG. 11 depicts a schematic perspective view of an illustrative manifold;

FIG. 12 depicts a schematic exploded view of an illustrative manifold assembly;

FIG. 13 depicts a schematic perspective view of an illustrative manifold assembly;

FIG. 14 depicts a schematic exploded view of the illustrative manifold assembly depicted in FIG. 13;

FIG. 15 is a schematic perspective view of the illustrative manifold assembly depicted in FIG. 13 coupled to a wall of a fluid source; and

FIG. 16 is a schematic cross-section view of the illustrative manifold assembly depicted in FIG. 13; and

FIGS. 17A and 17B depict flows through the illustrative manifold assembly depicted in FIG. 13, when the manifold assembly is in a first position and a second position.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices, systems, assemblies, and/or related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

An endoscope is used in performing diagnostic and/or therapeutic treatments by inserting an elongated shaft of the endoscope into a subject to observe a part to be examined within a body cavity of the subject and, if necessary, inserting a treatment instrument/tool into a working channel in the elongated shaft of the endoscope. Such endoscopes or endoscope systems may include a fluid/lens wash capability, or the like, configured to feed fluid, such as gas (e.g., air, CO₂), to an end of the endoscope for insufflating the inside of the subject at a target site. Lens wash features may provide sterilized water at relatively high pressure to spray across and clear debris from a camera lens of the endoscope. In order to rinse the target site of the subject, separate from the air/water feed capability, endoscopes or endoscope systems may have an irrigation capability that provides lower pressure, higher volume water, supplied via a pump (e.g., a peristaltic pump) to the target site in order to clear the field of view for observation and treatment. A water source (e.g., a fluid source) for lens wash and/or irrigation features may include one or more fluid reservoirs having tubing and cap assemblies that create a plumbing circuit in connection with the endoscope channels, valving, and/or connectors to accomplish the gas and water functions described.

Such tubing and cap assemblies may be available in various configurations, which may include a water bottle, a cap fitted for the specific bottle, and an array of tubing that is extendable through openings in the cap. The tubing typically is arranged to accommodate a specific configuration of endoscope fittings and valving, which does not tend to be modular or optional. In some cases, one or more connectors may be utilized to connect tubing for irrigation, lens wash, and/or insufflation features to an endoscope umbilical in fluid communication with working channels of the endoscope.

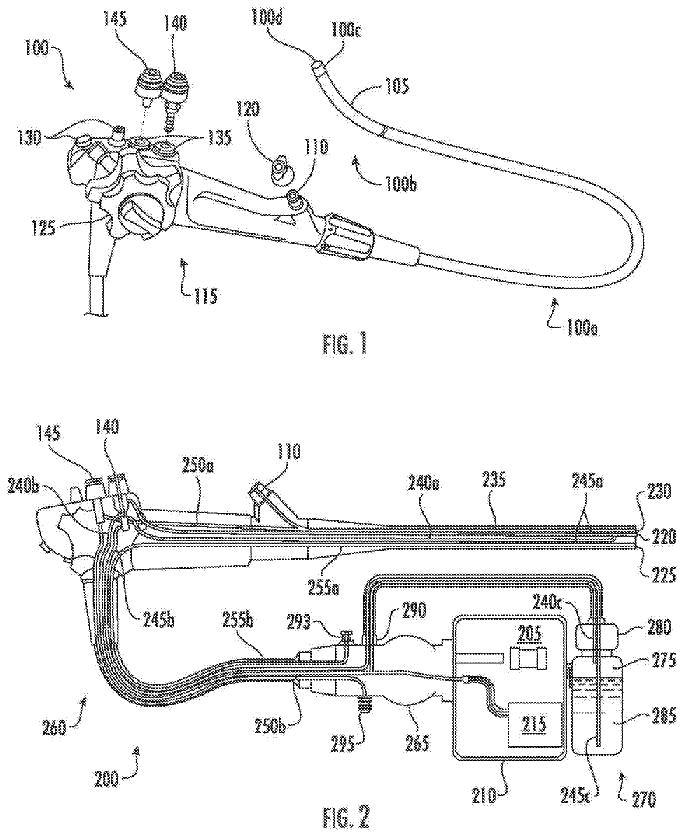

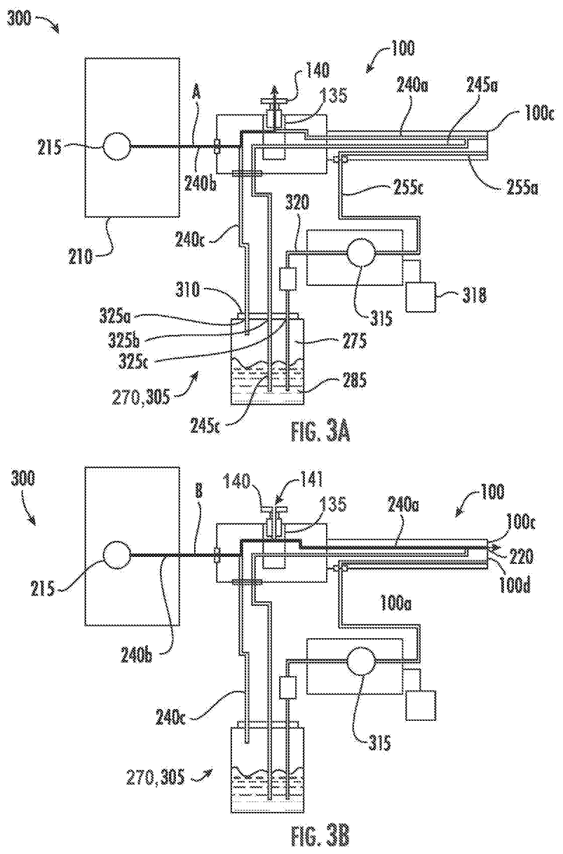

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient). Details of the endoscope 100 and the endoscope system 200 may be more fully described in U.S. Patent Application Publication No. 2022/0192479 A1, filed on Dec. 21, 2021, and titled TUBING ASSEMBLIES AND METHODS FOR FLUID DELIVERY, which is hereby incorporated by reference in its entirety for all purposes.

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., a lamp) may be located in a video processing unit 210 that processes signals input from the imager and outputs processed video signals to a video monitor (not shown) for viewing. The video processing unit 210 may also serve as a component of an air/water feed circuit by housing a pressurizing or air pump 215, such as an air feed pump, in the unit 210. Other suitable pumps for the air/water feed circuit are contemplated.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the subject at the treatment area and for supplying water to wash a lens covering the imager. An irrigation opening 225 in the end face 100d supplies irrigation fluid to the treatment area of the subject. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the flexible bending portion 105 (e.g., one knob may control up-down steering and another knob may control left-right steering). A plurality of video switches 130 for remotely operating the video processing unit 210 may be arranged on a proximal end side of the operating handle 115.

The operating handle 115 may be provided with dual valve locations 135. One of the valve locations 135 may receive a gas/water valve 140 for operating an insufflating gas and lens water feed operation. A gas supply line 240a and a lens wash line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 may have a gas (e.g., air or CO₂) feed line 240b, a lens wash feed line 245b, a suction feed line 250b, an irrigation feed line 255b, a light guide (not shown), an electrical signal cable (not shown), and/or other suitable lines, guides, and/or cables. The connector portion 265 when plugged into the video processing unit 210 connects the light source 205 in the video processing unit with the light guide. The light guide may run along the umbilical 260 and the length of the endoscope shaft 100a to transmit light to the distal tip 100c of the endoscope 100. The connector portion 265, when plugged into the video processing unit 210, may also connect the air pump 215 to the gas feed line 240b in the umbilical 260.

A fluid source, such as a fluid or water reservoir or container 270 (e.g., water bottle, bag, etc.) and/or other suitable fluid source, may be fluidly connected to the endoscope 100 through the connector portion 265 and the umbilical 260. A length of gas supply tubing 240c may pass from one end positioned in an air gap or gas 275 between the top 280 (e.g., a bottle cap, lid, closure, cover, etc.) of the reservoir 270 and the remaining water 285 (e.g., the remaining water 285) in the reservoir to a connector 290 on the outside of the connector portion 265. The gas feed line 240b from the umbilical 260 branches in the connector portion 265 to fluidly communicate with the gas supply tubing 240c at the detachable connector 290, as well as the air pump 215. A length of lens wash tubing 245c, with one end positioned at the bottom of the reservoir 270, may pass through the top 280 of the reservoir 270 to the same detachable connector 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some configurations, irrigation water may be supplied via a pump (e.g., peristaltic pump) from a water source (not shown) independent from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

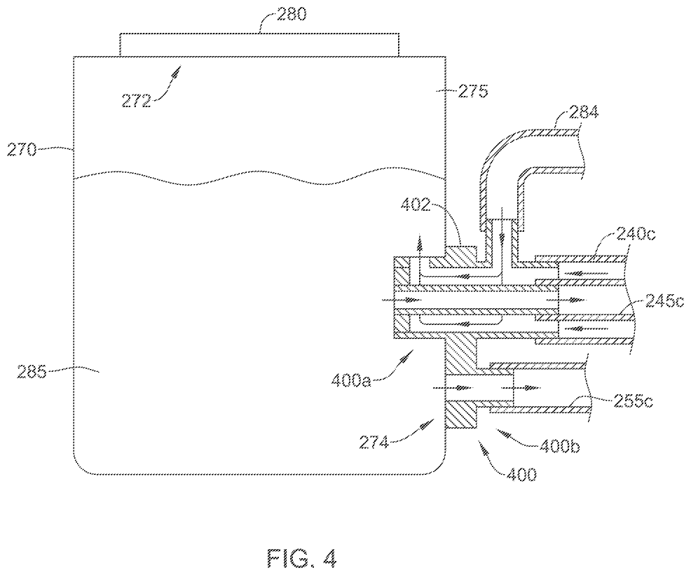

The gas supply tubing 240c and the lens wash tubing 245c may be combined in a coaxial relationship, but this is not required. In one example, the gas supply tubing 240c may define a lumen that is sufficiently large in diameter to encompass a smaller diameter lens wash tubing 245c, coaxially received within the gas supply tubing, as well as provide air to the water source in an annular space surrounding the lens wash tubing to pressurize the water reservoir (e.g., gas tubing 240c and lens wash tubing 245c as configured in the connector 290 depicted in FIG. 4). The lens wash tubing 245c may be configured to exit the lumen defined by the coaxial gas supply tubing in any suitable sealed manner, such as for example an aperture, fitting, collar, and/or the link for the purpose of transitioning from the coaxial arrangement to a side-by-side arrangement of the detachable gas/lens wash connection to the endoscope connector portion 265.

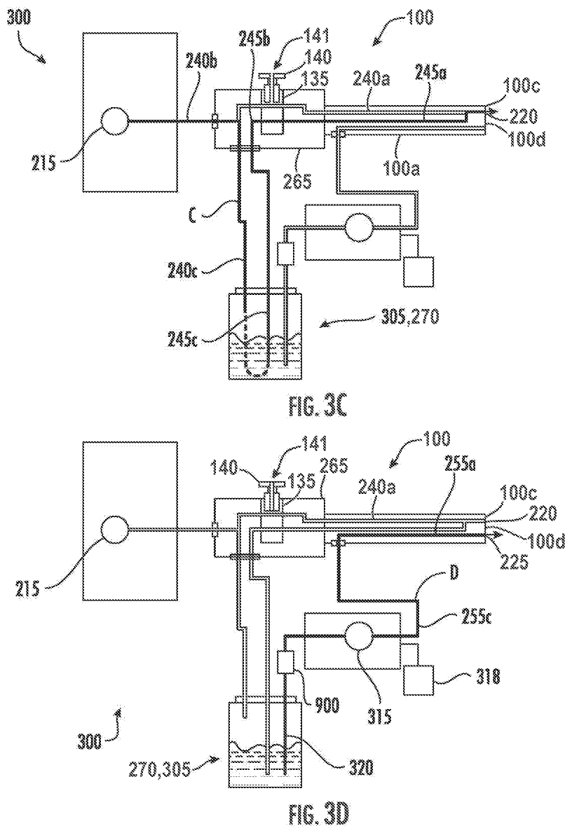

FIGS. 3A-3D are schematic views illustrating an operation of an embodiment of an endoscope system 300 (e.g., a medical device assembly), which may be similar or dissimilar to the endoscope system 200, where the supply tubing for irrigation and lens wash are connected to and drawn from a single water reservoir 270, 305 and/or other suitable fluid source. The hybrid system 300 may include the single water reservoir 270, 305, a cover or cap 310 to cover an opening of the reservoir, gas supply tubing 240c, lens wash tubing 245c, irrigation pump 315 that may be in communication with foot switch 318 or other suitable switch, upstream irrigation tubing 255c, 320, and downstream irrigation supply tubing 255c.

The cap 310 may be configured to attach in a seal-tight manner to the water reservoir 270, 305 by a threaded arrangement and/or other suitable coupling mechanism. The cap 310 may include a gasket to seal the cap 310 to the reservoir 270, 305. The gasket may be an O-ring, flange, collar, and/or the like and can be formed of any suitable material. A number of through-openings (325a, 325b, 325c) in the cap 310 may be provided to receive, respectively, the gas supply tubing 240c, lens wash tubing 245c, and upstream irrigation supply tubing 320, but this is not required. In FIGS. 3A-3D, the system depicted includes separate tubing for gas supply, lens wash, and irrigation.

In other embodiments, the gas supply tubing 240c and lens wash tubing 245c may be combined in a coaxial arrangement. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir. The lens wash tubing 245c may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265 (e.g., FIG. 2).

In various embodiments, different configurations of valving may be incorporated into the tubing of the system 200, 300. For example, an in-flow check valve may be disposed in the path of the gas supply tubing 240c to help prevent liquid backflow into the air pump 215. In this manner, pressure building within the water reservoir 270, 305 may create a pressure difference between the water reservoir 270, 305 and the gas supply tubing 240c helping to maintain a positive pressure in the water reservoir 270, 305 even when large amounts of water may be removed from the water source during the irrigation function. This arrangement may compensate for any time lag in air being delivered from the air pump 215 to the water reservoir 270, 305, which might otherwise cause a negative pressure vacuum in the water reservoir 270, 305. Similarly, an out-flow check valve, such as a one-way valve, may be incorporated in the lens wash tubing 245c, upstream irrigation supply tubing 320, and/or downstream irrigation supply tubing 255c to help prevent backflow of water from either or both of the lens wash and irrigation tubing in the event of a negative pressure situation, as described.

More generally, in some configurations, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check, a flapper valve, and/or other suitable check valve. Accordingly, a check valve as used herein may be meant to be separate and distinct from an active valve that is operated in a binary manner as an on/off valve or switch to allow flow to be turned on or allow flow to be turned off (e.g., a stop cock valve, solenoid valve, peristaltic pump, blow off valve).

During operation of the system of FIGS. 3A-3D, a flow of water for irrigation may be achieved by operating the irrigation pump 315 via the foot switch 318 and/or other suitable actuation mechanism. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 270, 305, the pressure in the system may be controlled to maintain the lens wash tubing 245c at substantially the pressure necessary to accomplish a lower flow rate lens wash, while compensating for reduced pressure in the water reservoir 270, 305 due to supplying a high flow rate irrigation. When pressure is reduced in the water reservoir 270, 305 by use of the lens wash function, the irrigation function, or both functions simultaneously, the reduced pressure may be compensated for by the air pump 215 via the gas supply tubing 240c.

Flow paths in the schematic set-ups depicted in each of FIGS. 3A-3D have been highlighted to show the different flow paths possible with the hybrid system 300 having supply tubing (e.g., irrigation tubing 255c, 320, lens wash tubing 245c, and/or other suitable tubing) connected to and drawn from the single water reservoir 270, 305. Not all features depicted in each of FIGS. 3A-3D are labeled with reference numerals in each of FIGS. 3A-3D for clarity purposes, but similarly depicted features in FIGS. 3A-3D should be understood to be referring to a same or similar feature in each of FIGS. 3A-3D.

As shown in FIG. 3A, the endoscope 100 may be in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b (e.g., in the umbilical 260 via the connector portion 265, as depicted in FIG. 2) and through the gas/water valve 140 to atmosphere. Because the system is open at the vent hole in the gas/water valve 140, there is no build up to pressurize the water reservoir 270, 305 and consequently no water is pushed through the lens wash tubing 245c.

As shown in FIG. 3B, the endoscope 100 may be in a gas delivery state with the gas/water valve 140 in a first position. When gas is called for at the distal tip 100c, for example, to clean the end face 100d of the distal tip 100c or insufflate the patient body in the treatment area, the user may close off a vent hole 141 in the gas/water valve 140 with a thumb, finger, or the like (first position). In this state, gas (pressure) may be delivered along path B from the air pump 215 and flowed through the gas feed line 240b (e.g., in the umbilical 260 via the connector portion 265, as depicted in FIG. 2). The gas may continue through the gas/water valve 140 to the gas supply line 240a in the endoscope shaft 100a and out the gas/lens wash nozzle 220 at the distal tip 100c. There is no build up to pressurize the water reservoir since the system is open at the gas/lens wash nozzle 220, and consequently no liquid is pushed through the lens wash tubing 245c.

As shown in FIG. 3C, the endoscope 100 may be in a lens wash delivery state with the gas/water valve 140 in a second position. When lens wash is called for at the distal tip 100c, for example, to clean the end face 100d of the distal tip 100c, the user, keeping the vent hole 141 in the gas/water valve 140 closed off, depresses the valve 140 to its furthest point in the valve well 135. The second position blocks off the gas supply to both atmosphere and the gas supply line 240a in the endoscope 100, and opens up the gas/water valve 140 to allow lens wash water to pass through to the lens wash line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 270, 305. The gas (pressure) pressurizes the surface of the remaining water 285 in the reservoir 270, 305 and pushes water up the lens wash tubing 245c (e.g., to the connector portion 265 of the umbilical 26, as depicted in FIG. 2. The pressurized lens wash water may be pushed further through the lens wash feed line 245b and through the gas/water valve 140. Because the system 300 is closed, gas pressure may be allowed to build and maintain a calibrated pressure level in the water reservoir 270, 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user may activate the irrigation pump 315 (e.g., by depressing foot switch 318 or other suitable actuation mechanism) to deliver water or other liquid from the reservoir 270, 305 along path D. With the pump 315 activated, water is sucked out of the water reservoir 270, 305 through the upstream irrigation supply tubing 255c, 320 and pumped along the downstream irrigation supply tubing 255c to the connector portion 265. The irrigation pump head pressure pushes the irrigation water further through the irrigation feed line 255b (e.g., extending through the umbilical 260), through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

Coupling of tubing assemblies of the endoscope system 200 to fluid sources can be burdensome. For example, at the beginning of a day in an endoscope or other scope procedure room, an endoscope must be coupled to associated pieces of tubing that enable irrigation, lens wash, insufflation, etc., and this tubing may need to be coupled to one or more fluid sources. Further, the coupling of tubing assemblies may need to be repeated several times during the day for each new procedure and/or to replace fluid sources. As such, creating a manifold or bulkhead that is configured as a single location at which tubing of an endoscope assembly may couple to one or more fluid sources (e.g., gas and water fluid sources), may reduce a number of steps needed to fluidly connect tubing with a fluid source. In one example, a manifold or bulkhead may be configured to releasably couple to a plurality of tubes of an endoscope assembly and releasably couple with a liquid container or source, but this is not required.

FIG. 4 is a schematic view of an illustrative manifold 400 (e.g., shown schematically in cross-section) coupled to the water reservoir 270 and tubing of a tubing assembly configured to couple with an umbilical of an endoscope system 200. In some cases, the manifold 400 may be a bulkhead fitting or component between the water reservoir 270 and tubing, but this is not required.

The manifold 400 may be configured to bridge a wall of the water reservoir 270 (e.g., the sidewall extending between a first end and a second end of the water reservoir 270 or other suitable wall) between the inside and outside of the water reservoir 270 and act as a conduit for allowing fluids (e.g., gasses and/or liquids) to leave and/or enter the water reservoir 270. The manifold 400 may facilitate a user connecting tubing at a single location, while reducing the number of parts utilized for connecting tubing to the water reservoir 270. Further, in some cases, the manifold 400 may reduce a likelihood of contamination of water by not requiring tubing connected to the manifold 400 to enter an interior of the water reservoir 270 (e.g., enter into the water 285 and/or the gas 275, such as air, CO₂, etc. in the water reservoir 270).

The manifold 400 may be coupled to various tubing sets that facilitates using an endoscope 100 for lens wash, irrigation, insufflation, etc. As depicted in FIG. 4, the manifold 400 may be coupled to wash lens tubing 245c, gas or air supply tubing 240c, irrigation tubing 255c, carbon dioxide tubing (CO₂) 275, and/or other suitable tubing of the tubing assembly. In some cases, the tubing may include, a single lumen and/or multiple coaxial and/or parallel lumens (e.g., the lens wash tubing 245c and the gas or air tubing 240c may be configured in a coaxial relationship). The manifold 400 may be configured to receive gas from tubing coupled to the manifold to attain and/or maintain a desired pressure in the water reservoir 270 and output water from the water reservoir 270 to the tubing coupled to the manifold 400.

The manifold 400 may be coupled to the water reservoir 270 at any suitable location. For example, the manifold 400 may be coupled to the water reservoir 270 along a top or cap 280, a side wall extending between the top and bottom of the water reservoir 270 (e.g., as depicted in FIG. 4 or otherwise), and/or along the bottom of the reservoir 270. In one example, the manifold 400 may be positioned in or may extend through a sidewall of the water reservoir (e.g., a side wall of the container of the water reservoir 270) to facilitate placing the openings of the manifold configured to receive water near or proximate a bottom of the water reservoir 270.

The water reservoir 270 may have a first opening 272, a second opening 274, and/or other suitable number of openings. In the schematic example depicted in FIG. 4, the water reservoir 270 may include the first opening 272 at a top of the water reservoir 270 or other suitable location, which may be covered or sealed by the cap or top 280, and the second opening 274, which may be configured to receive the manifold 400, as depicted in FIG. 4, may be located in the side wall of the water reservoir 270 proximate a bottom of the water reservoir 270.

The manifold 400 may be coupled to the water reservoir 270 in any suitable manner. Example techniques for coupling the manifold 400 to the water reservoir 270 may include, but are not limited to, using adhesives, silicone adhesives, a friction fit, a threaded coupling with the water reservoir, a snap connection between components of the manifold 400, a threaded connection between components of the manifold 400, a luer lock connection between components of the manifold 400, a heat pressed seal, and/or other suitable types of connections. The manifold 400 may be coupled to a single use water reservoir 270 and/or a reusable (e.g., refillable) water reservoir 270, as desired. In some cases, the coupling between the water reservoir 270 and the manifold 400 may create a hermetic seal.

The manifold 400 may include a central portion 402, which may be configured to divide the manifold into a first portion 400a (e.g., an interior portion) and a second portion 400b (e.g., an exterior portion). The first portion 400a of the manifold 400 may be configured to be positioned within the water reservoir 270 and the second portion 400b may be configured to remain exterior of the water reservoir, as depicted in FIG. 4. The central portion 402 may be an interface configured to couple to the interior and/or the exterior of the water reservoir 270 and/or otherwise facilitate sealing the opening 274.

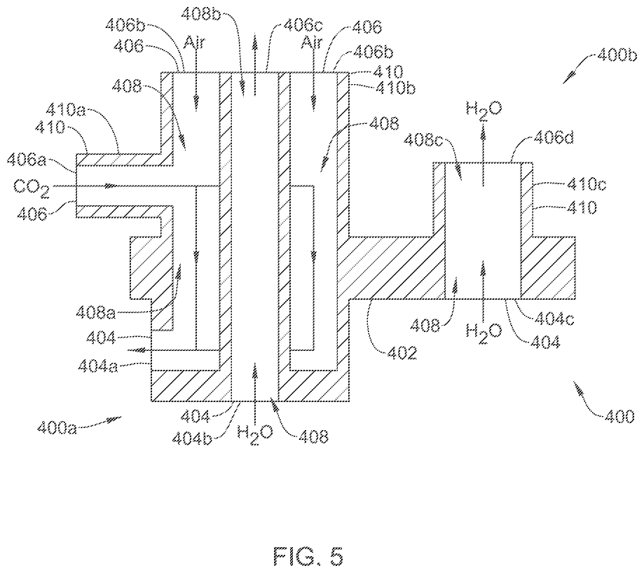

FIG. 5 depicts a schematic cross-sectional view of a manifold 400. Although the manifold 400 depicted in FIG. 5 is depicted as being formed monolithically from a single material, the manifold 400 may be formed from two or more materials and/or components.

The manifold 400 may include one or more openings 404 on the first portion 400a and one or more openings 406 on the second side 400b of the manifold 400, where the interior openings 404 and the exterior openings 406 may be fluidly coupled with one or more through holes 408 (e.g., pathways, lumens, etc.). In an example depicted in FIG. 5, the manifold 400 may include a first opening 404a, a second opening 404b, and a third opening 404c on the first portion 400a of the manifold 400, and a first opening 406a, a second opening 406b, a third opening 406c, and a fourth opening 406d on the second portion 400b of the manifold 400. A first through hole 408a may fluidly couple (e.g., be in fluid communication with) the first opening 404a on the first portion 400a with the first opening 406a and the second opening 406b on the second portion 400b of the manifold 400. A second through hole 408b may fluidly couple (e.g., be in fluid communication with) the second opening 404b on the first portion 400a with the third opening 406c on the second portion 400b of the manifold 400. A third through hole 408c may fluidly couple (e.g., be in fluid communication with) the third opening 404c on the first portion 404a with the fourth opening 406d on the second portion 400b of the manifold 400. The first through hole 408a and the second through 408b are depicted in FIG. 5 as being co-axial, but this is not required and the through holes 408a, 408b may be configured in one or more other suitable manners. Further, although first and second openings 406a, 406b on the second portion 400b of the manifold 400 fluidly communicate with a single through hole, each of the first and second opening 406a, 406b may have separate through holes that couple to a single opening or multiple openings on the first portion 400a of the manifold 400. Other suitable configurations of the openings 404, 406 and through holes 408 of the manifold 400 are contemplated.

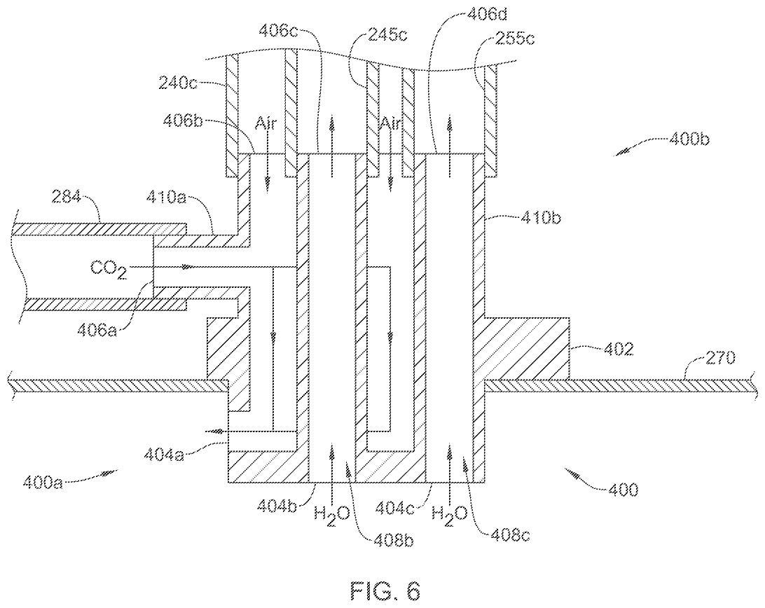

The second side 400b of the manifold 400 may include any suitable number of ports 410 configured to couple with tubing, connectors, and/or other components of the endoscope system 200. In one example configuration, the second portion 400b of the manifold 400 may include a first port 410a at the first opening 406a, a second port 410b at the second and third openings 406b, 406c, and a third port 410c at the fourth opening 406d. In other example configurations, the manifold 400 may include a single port configured to couple to a complex tubing, two ports (e.g., see the configuration of the manifold 400 depicted in FIG. 6) or more than three ports, as desired. The ports 410 may be configured to couple to tubing of the endoscope assembly 200 in any suitable manner including, but not limited to, friction fits, clamps, clips, threads, prongs, and/or other suitable connection techniques.

The fluid flows may pass through the openings 404, 406 and the through holes 408 of the manifold 400 in any suitable manner. In one example set up of the manifold 400, pressurized CO₂ may enter the first opening 406a on the second side 400b of the manifold 400, travel through the first through hole 408a, and exit through the first opening 404a on the first portion 400a of the manifold 400. In another example, pressurized air may enter the second opening 406b on the second side 400b of the manifold 400, travel through the first through hole 408a, and exit through the first opening 404a. In operation, only one of air and CO₂ may be pumped into the water reservoir 270 at a time and/or both air and CO₂ may be pumped into the water reservoir 270. In a further example, when the water reservoir 270 is sufficiently pressurized (e.g., via the CO₂ and/or the air received in the water reservoir 270), water (H₂O) may be forced out of the water reservoir 270 and into the second opening 404b on the first portion 400a of the manifold 400, through the second through hole 408b, and out of the third opening 406c on the second side 400b of the manifold 400. In another example, when the water reservoir 270 is sufficiently pressurized (e.g., via the CO₂ and/or the air received in the water reservoir 270) and/or in response to a downstream pump, water (H₂O) may be forced out of the water reservoir 270 and into the third opening 404c on the first portion 400a of the manifold 400, through the third through hole 408c, and out of the fourth opening 406d on the second side 400b of the manifold 400. Other suitable setups of the manifold 400 relative gasses and liquid passing through the manifold 400 are contemplated.

FIG. 6 depicts a schematic cross-sectional view of an illustrative configuration of the manifold 400 coupled to and/or engaging a wall of the water reservoir 270, where the second portion 400b of the manifold 400 has two ports 410a, 410b. The first port 410a may be configured to couple to CO₂ tubing 275 and the second port 410b may be configured to couple to a multi-lumen tubing having air tubing 240c, lens wash tubing 245c, and irrigation supply tubing 255c, but it is contemplated that the multi-lumen tubing may include additional or alternative tubing therein. The tubing depicted in FIG. 6 includes co-axial tubing and a parallel tubing, where the irrigation supply tubing 255c runs parallel to the co-axial lens wash tubing 245c and air supply tubing 240c. Although the manifold 400 depicted in FIG. 6 is depicted as being formed monolithically from a single material, the manifold 400 may be formed from two or more materials and/or components.

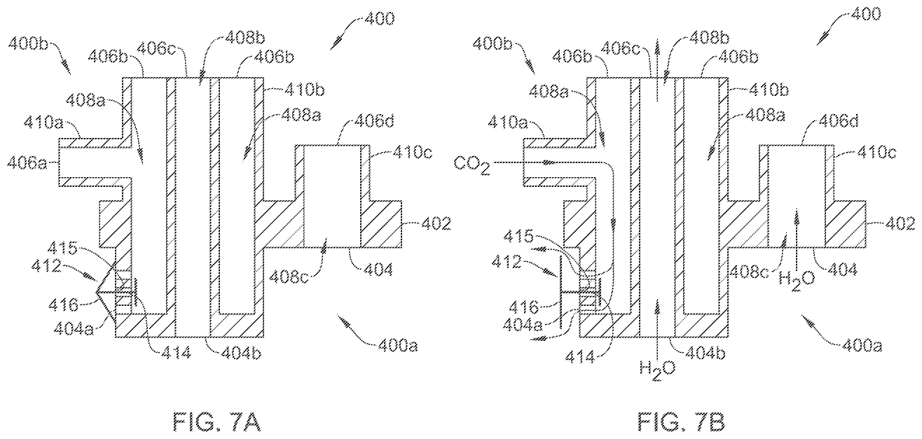

FIG. 7A and FIG. 7B schematically depict a cross-sectional view of an illustrative configuration of the manifold 400, with a valve 412 (e.g., a one-way valve or other suitable valve) at the first opening 404a. FIG. 7A depicts the manifold 400 with the valve 412 in a closed or resting position (e.g., a first position). FIG. 7B depicts the manifold 400 with the valve 412 in an opened or actuated position (e.g., a second position).

The valve 412 may be any suitable type of valve. Example types of suitable valves include, but are not limited to, a one-way valve, a check valve, a ball check valve, a diaphragm check valve, an umbrella valve, a swing check valve, a tilting disc check valve, a flapper valve, a stop-check valve, a lift-check valve, an in-line check valve, a duckbill valve, a pneumatic non-return valve, a reed valve, a flow check, a spring-loaded valve, flapper valve, a kinking system, and/or other suitable valves.

In one illustrative example of a valve 412, as depicted in FIGS. 7A and 7B, the valve 412 may be include a connector 414 and a resilient cover 416. The connector 414 may be coupled to the resilient cover 416 and may extend through a wall (e.g., where the wall defines an opening 415 through which the connector 414 extends) of the first portion 400a of the manifold 400 defining the first opening 404a. When the connector 414 is securing the valve 412 to the first portion 400a of the manifold 400, the cover 416 may extend over the first opening 404a in a resting position and prevent flow from the water reservoir 270 from entering the first opening 404a.

In operation, when the pressure in the first through hole 408a has not reached a desired threshold pressure, the valve cover 416 remains in a closed or resting position to prevent fluid from the water reservoir 270 from entering the first through hole 408a through the first opening 404a, as depicted in FIG. 7A. When pressure in the first through hole 408b reaches or goes beyond the threshold pressure, the cover 416 may automatically adjust to an opened position to allow fluid (e.g., CO₂ in the depicted example) to pass through the first opening 404a. Once the pressure in the first through hole 408a returns to a pressure that has not reached or gone beyond the threshold pressure, the cover 416 may automatically return to its closed or resting state to prevent fluid from the water reservoir 270 from entering the first opening 404a and the first through hole 408a. While the cover 416 is in the opened or actuated position, fluid passing through the first opening 404a from the first through hole 408a is at a sufficient pressure and/or flow rate so as to prevent fluid from entering the first opening 404a from the water reservoir 270 and the cover 416 may be configured to close before the fluid from the first through hole 408a drops below a pressure or flow rate needed to prevent fluid from entering the first opening 404a from the water reservoir 270.

The cover 416 may be made from any suitable material configured to adjust positions at a desired difference in pressure across the valve 412 and/or the first opening 404a. In one example, the cover 416 may be made from silicone, elastomers, thermoplastic elastomers (TPE), rubberized material, and/or other suitable materials.

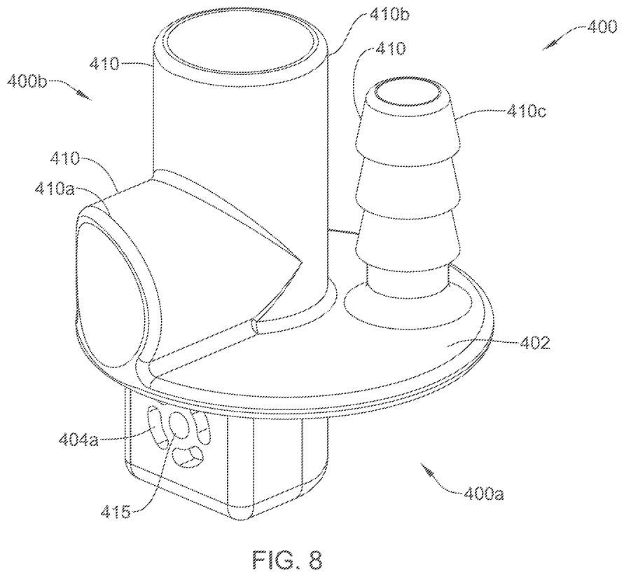

FIGS. 8-10 are schematic view of a configuration of the manifold 400 with two openings on the first portion 400a and three ports at respective openings on the second side of manifold 400. The configuration of the manifold 400 depicted in FIGS. 8-10 may allow for all or several tubing lines of the endoscope system 200 to extend to or from a single location (e.g., at a bulkhead) at or proximate a water reservoir. The manifold 400 may include any suitable number of ports. In one example, the manifold 400 may include the first port 410a for coupling with CO₂ (or air insufflation) tubing, the second port 410b for coupling with lens wash tubing, and the third port 410c for coupling with irrigation tubing. Other suitable port configurations are contemplated for coupling with additional and/or alternative tubing lines.

As depicted in FIG. 8, the first port 410a, the second port 410b, and the third port 410c may extend from the central portion 402. In the configuration of the manifold 400 depicted in FIGS. 8-10, the central portion 402 of the manifold 400 may be configured to couple to a fluid reservoir (e.g., the water reservoir 270, 305 and/or other suitable fluid reservoir). In one example, adhesive, silicone adhesives, and/or other suitable coupling material may be used to couple or otherwise connect a side of the central portion 402 facing the first portion 400a of the manifold 400 to a wall of the fluid reservoir defining an opening configured to receive the first portion 400a of the manifold 400. The coupling between the manifold 400 and the fluid reservoir may create a hermetic seal, as discussed above.

One or more of the ports 410a, 410b, 410c may include fittings configured to secure a tubing thereto. Example fittings include, but are not limited to, barbs, ribs, ridges, tapered surfaces, a ball-detent, luer lock, and/or other suitable fittings. In one example, one or more of the ports 410a, 410b, 410c may include tapered ridges (e.g., the third port 410c may include tapered ridges, as depicted in FIG. 8), where the tapered ridges may be configured to facilitate receiving tubing and preventing unintentional separation of the tubing and the port.

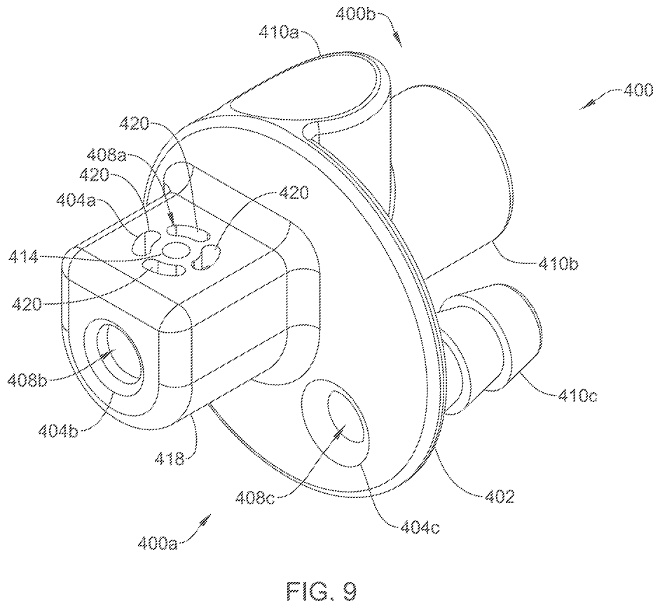

FIG. 9 depicts a schematic perspective view of the first portion 400a of the manifold 400. In some cases, the first portion 400a of the manifold 400 may include an extension 418 that may extend outward from a side of the central portion 402 facing the first potion 400a. In one configuration of the extension 418, the extension 418 may include and/or entirely or at least partially define the first interior opening 404a and the second interior opening 404b, where the first interior opening 404a is in fluid communication with the first through hole 408a and the second interior opening 404b is in fluid communication with the second through hole 408b. The third interior opening 404c may be defined in the side of the central portion 402 facing the first portion 404a of the manifold, where the third opening 404c may be in fluid communication with the third through hole 408c. Alternatively or additionally, all or some of the interior openings 404a, 404b, 404c and/or other openings may be formed in the extension 418 and/or in the central portion 402.

As depicted in FIG. 9, the first interior opening 404a may include a plurality of sub-openings 420 with material of the extension 418 extending between the sub-openings 420. Although not required, such a configuration of the first interior opening 404a may facilitate the extension 418 defining the opening 415 through which a valve controlling flow through the first interior opening 404 may extend (e.g., the connector 414 of the valve 412 may extend through the opening 415). Other suitable configurations of the first interior opening 404a and/or the extension 418 are contemplated to facilitate utilizing a valve to control flow through the first interior opening 404a.

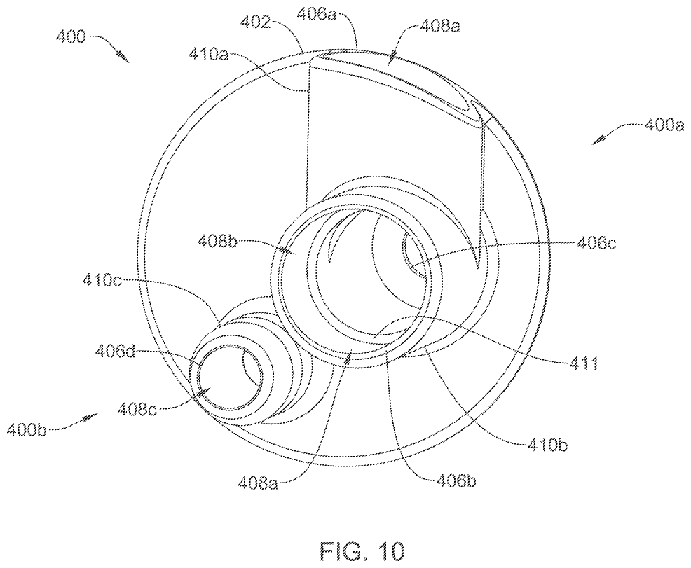

FIG. 10 depicts a schematic perspective view of the second portion 400b of the manifold 400. In some cases, the second portion 400b of the manifold 400 may include the first exterior opening 406a defined at or by the first port 410a, the second and third exterior openings 406b, 406c defined at or by the second port 410b, and the fourth exterior opening 406d defined at or by the third port 410c, but other suitable configurations are contemplated. The first exterior opening 406a and the second exterior opening 406b may be in fluid communication with the first through hole 408a and the first interior opening 404a to pass air and/or CO₂ into the fluid reservoir so as to achieve a desired pressure within the fluid reservoir to which the manifold 400 is coupled. The third exterior opening 406c may be in fluid communication with the second through hole 408b and the second interior opening 404b to receive pressurized water or other suitable liquid from the fluid reservoir in response to pressure within the fluid reservoir reaching and maintaining a liquid actuation pressure. The fourth exterior opening 406d may be in fluid communication with the third through hole 408c and the third interior opening 404c to draw liquid out of or receive pressurized liquid from the fluid reservoir. In some cases, a downstream pump (e.g., a peristaltic pump and/or other suitable type of pump) may draw liquid from the fluid reservoir through the fourth exterior opening 406d, but this is not required.

Further, the ports 410a, 410b, 410c and/or other suitable ports may include one or more features thereon to indicate which tubes are to be connected to which ports. In one example, the ports may include color coding that is configure to match color coding on the tubing, where tubing and ports having the same or similar color are to be coupled to one another. In another example, each of the ports may be sized to as to only allow connection with an intended tubing. The ports, however, may include other suitable tube-indicating features, as desired.

The ports 410a, 410b, 410c and/or other suitable ports may be configured to connect to tubes in any suitable manner. In one example, the ports 410a and/or 410b may include a ledge 411 for abutting an end of tubing received in the port (e.g., as shown in the second port 410b in FIG. 10). Alternatively or additionally, tubing may be positioned over the ports and/or coupled to the ports in one or more other suitable manners.

The configuration of the ports 410a, 410b, and 410c depicted in FIGS. 8-10 may have several benefits. In one example, the positioning of the ports 410a, 410b, 410c may facilitate fluidly coupling tubing to the fluid reservoir without requiring the tubing to be disposed inside of the fluid reservoir or a container of the fluid reservoir. In another example, the positioning of the second interior opening 404b and the third opening 404c may facilitate collecting as much liquid from the fluid reservoir as possible when the manifold 400 is positioned proximate a bottom of the fluid reservoir.

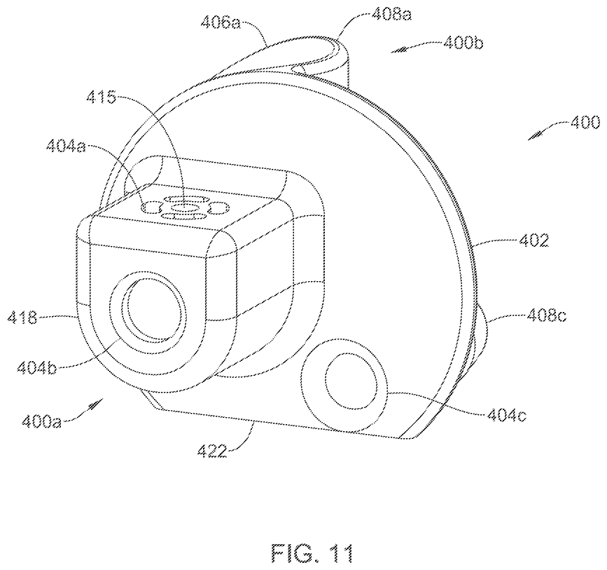

FIG. 11 depicts a schematic perspective view of an illustrative configuration of the manifold 400 having a contoured surface 422 defining a perimeter of the central portion 402. Although the contoured surface 422 may take on any desired configuration, the contoured surface 422 depicted in FIG. 11 may be a straight line extending along the perimeter of the central portion 402 that creates a flat surface to facilitate positioning the manifold 400 at or near a bottom of the fluid reservoir and to position the second and third openings 404b, 404c of the first portion 400a as near the bottom of the fluid reservoir as possible, while not interfering with a surface on which the fluid reservoir rests (e.g., such that the manifold does not extend below a bottom outer surface of the fluid reservoir). In some cases, the contoured surface 422 may be positioned at a location parallel to the first interior opening 404a and a valve controlling fluid flow through the first interior opening 404a (e.g., the valve 412 and/or other suitable valve) and at an opposite side of the manifold from which the first opening 404a faces.

As discussed above, the manifold 400 may be configured as a single component and/or may be multiple components configured to couple together. In some case, when the manifold 400 is configured from multiple components, the multiple components may be coupled or connected to one another in a manner that secures or couples the manifold 400 to the fluid reservoir. In one example, the manifold 400 may be formed from at least a first component and a second component, where the first and second component are configured to couple to one another to form the manifold 400. When so configured, a first component may be inserted into the fluid reservoir through the opening thereof that the manifold 400 is configured to extend and/or other suitable opening. Once inside the fluid reservoir, the first component may couple to a second component of the manifold 400 through the opening of the fluid reservoir (e.g., in the wall of the fluid reservoir) and seal the opening such that fluid can only leave or enter through the manifold 400.

When configured from multiple components, the manifold 400 may include features that facilitate maintaining a fluid-tight seal between the multiple components. Example features that facilitate maintaining a fluid-tight seal between multiple components include, but are not limited to, resilient o-rings, resilient material layers between components, adhesives, etc.

The components of the manifold 400 may be coupled to one another in any suitable manner via a connector (e.g., a connection technique and/or connection components). Example suitable connection techniques include, but are not limited to, threaded connections, luer lock connections, snap connections, friction fit connections, ball-detent connections, adhesive, and/or other suitable connection techniques.

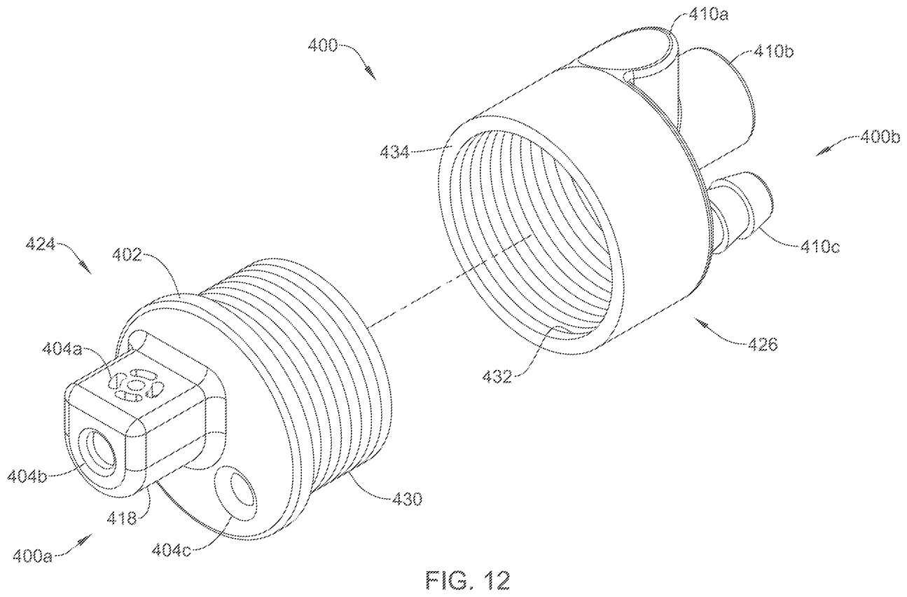

FIG. 12 depicts a schematic perspective exploded view of a configuration of the manifold 400, where the manifold 400 has a first component 424 configured to couple with a second component 426 via connector. As depicted in FIG. 12, the first component 424 has a threaded male component 430 configured to engage or connect with a threaded female component 432, where the threaded components 430, 432 may collectively be a connector.

In operation, once the first component 424 has been inserted in the fluid reservoir, the threaded male component 430 may be positioned through the opening in the fluid reservoir. The second component 426 may be engaged with the first component 424 extending through the opening in the fluid reservoir and rotated such that the threads of the threaded female component 432 engage the threads of the threaded male component 430. When fully engaged, a surface of the central portion 402 that is facing the second component 426 may engage an inner surface of a wall of the manifold that is defining the opening through which the manifold 400 extends. Further, when fully engaged, a ledge 434 of the second component 426 facing the first component 424 may engage an outer surface of the fluid reservoir.

In some cases, the threaded male component 430 may be keyed with the threads of the threaded female component 432 such that when the first component and second component are fully coupled to one another about the opening in the fluid reservoir, inner through holes of the manifold 400 align with interior openings and exterior openings of the manifold. When the manifold 400 is coupled to fluid reservoir, but tubing has not been engaged with ports of the manifold 400, one or both of the first and second components 424, 426 of the manifold may be over-tightened or slightly loosened to misalign the interior and/or exterior openings relative to the through holes of the manifold 400 to prevent fluid from entering and/or exiting the fluid reservoir.

When the manifold 400 is formed from two or more components, one or both of the components may be replaceable and may be replaced due to wear, to change functionality of the manifold 400, and/or replaced for one or more other suitable reasons. In one example, the first component 424 may be secured to an interior surface of the fluid reservoir and the second components 426 may be replaced to change the functionality (e.g., configuration of ports, etc.) of the manifold 400 depending on needs of a procedure utilizing the endoscope in fluid communication with the fluid reservoir.

In another example configuration of a multiple component manifold 400, one or more of the components may be configured to have one or more smaller diameter portions that are configured to extend through smaller diameter holes in the fluid reservoir rather than a single, larger diameter component containing all of the through holes of the manifold 400 and configured to extend through a single larger diameter hole in the fluid reservoir. In such a configuration, the first component of the manifold 400 may have a smaller diameter male component (e.g., cylindrical posts or components having other suitable configurations) for each through hole of the manifold 400 and the second component of the manifold 400 may be configured to engage the smaller diameter male components extending through the smaller diameter openings in the fluid reservoir and seal of the smaller diameter openings. In such a configuration, the first component and the second component may be coupled via a silicon adhesive to facilitate preventing fluid leakage through the smaller diameter holes.

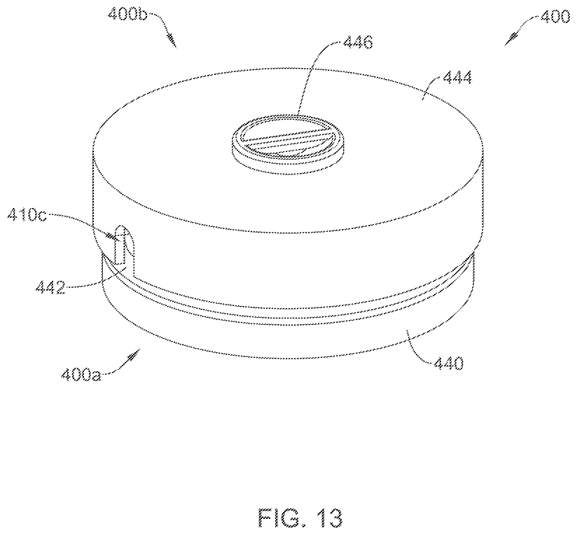

FIGS. 13-17 depict an illustrative configuration of the manifold 400 that allows tubing coupled to the manifold 400 to extend from the manifold 400 in a direction that is perpendicular to the manifold 400 and tangential to the fluid reservoir. In such a configuration, the tubing may exit the manifold 400 and be pointed directly in a desired path to be coupled with a pump (e.g., a peristaltic pump in the case of irrigation tubing and/or other suitable pump), an endoscope umbilical, and/or other feature of the endoscope system.

FIG. 13 depicts a schematic perspective view of an illustrative configuration of the manifold 400 configured to have coupled tubing extend perpendicularly therefrom. The manifold 400 depicted in FIG. 13 may include the first portion 400a and the second portion 400b and may be formed from a plurality of components. In one example, the manifold 400 may include, among other components, a base 440, an intermediate component 442, a cover 444, and an actuator 446. The actuator 446 may be accessible from the second portion 400b and may be actuated or otherwise adjusted to adjust a flow of fluid through the manifold 400 (e.g., adjust a flow of fluid through the first portion 400a and/or the second portion 400b), but this is not required.

As discussed in greater detail below, the manifold 400 of FIG. 13 may include one or more ports. As depicted in FIG. 13, the intermediate component 442 and the cover 444 cooperate to form the third port 410c configured to couple with irrigation tubing and/or other suitable tubing.

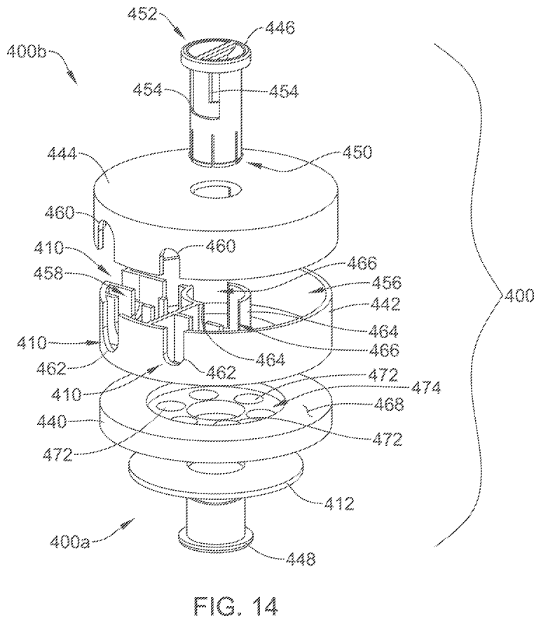

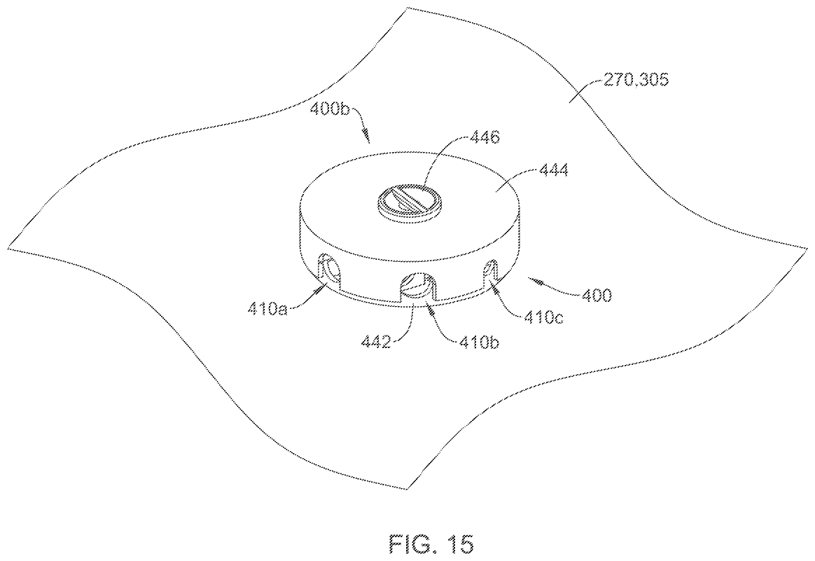

FIG. 14 depicts a schematic exploded view of the illustrative manifold 400 depicted in FIG. 13. As shown in FIG. 15, the manifold 400 may include, among other components, a clip connector 448, the valve 412, the base 440, the intermediate component 442, the cover 444, and the actuator 446. To couple the components of the manifold 400 together (e.g., together and around a wall of a fluid reservoir), the clip connector 448 may be inserted through the valve 412, the base 440, and into the intermediate component 442 and the actuator 446 may be inserted (e.g., in an opposite direction of a direction in which the clip connector 448 is inserted) through the cover 444, the intermediate component 442, the base component 440, the valve 412, and the clip connector 448, where the actuator 446 may engage the clip connector 448 to secure the components of the manifold 400 to one another. In the configuration of the manifold 400 depicted in FIG. 14, the actuator 446 and/or the clip connector 448 may be inserted through axially aligned central openings extending through the valve 412, the base 440, the intermediate component 442, and the cover 444.

The actuator 446 may include a clip feature 450 configured to extend through a central opening of the clip connector 448 and clip to the clip connector 448. When connected to the clip connector 448, the actuator 446 may couple the first portion 400a (e.g., clip connector 448, the valve 412, and the base 440) of the manifold 400 with the second portion 400b (e.g., the intermediate component 442 and the cover 444) of the manifold 400 and may be able to rotate freely. Although the actuator 446 is depicted with a clip feature 450, the actuator 446 and/or the clip connector 448 may be configured to engage in one or more other suitable manners, including, but not limited to, a threaded coupling, a luer lock coupling, a ball detent coupling, and/or other suitable engagement techniques that may allow the actuator 446 to rotate once engaged with or otherwise coupled with the clip connector 448.

The actuator 446 may be any suitable actuator 446 configured to facilitate coupling the components of the manifold 400 to one another and configured to adjust a flow of pressurized fluid (e.g., liquid) from the fluid reservoir to the manifold 400 and/or tubing. As discussed above, the actuator 446 may include a clip feature 450 at a first end of the actuator 446 and a user interface 452 at a second end of the actuator 446. The user interface 452 may facilitate a user interacting (e.g., rotating or otherwise interacting) with the actuator 446 to adjust a flow of liquid from the fluid reservoir through the manifold 400. Between the user interface 452 and the clip feature 450, the actuator 446 may include one or more windows 454. As depicted in FIG. 14, the actuator may include two windows, but other suitable number of windows may be utilized, as desired.

The cover 444 may be any suitable type of cover 444. In some cases, the cover 444 may be configured to at least partially define one or more chambers (e.g., a first chamber 456 and a second chamber 458, or other suitable chambers) of the manifold 400 with the intermediate component 442. Additionally or alternatively, the cover 444 may at least partially define ports 410 for engaging and/or receiving tubing. In one example, the cover 444 may include side openings 460 configured to align with side openings 462 of the intermediate component to form ports 410. Such an example configuration of the ports may facilitate receiving tubing or a fitting thereof, placing the tubing in the side openings 462 of the intermediate component 442 and then applying the cover 444 over the received and placed tubing to secure the tubing in place with the side openings 460 of the cover 444 engaging the received tubing and/or fittings of the tubing.

As discussed above, the intermediate component 442 may form or at least partially form one or more chambers. As depicted in FIG. 14, the intermediate component 442 may at least partially define the first chamber 456 and the second chamber 458. In some case, the first chamber 456 may be configure to receive liquid from the fluid reservoir that may enter the first chamber 456 through a central opening extending through the clip connector 448, a central opening of the actuator 446, one or more windows 454 of the actuator 446. The second chamber 458 may be configured to receive gases for delivery to the fluid reservoir through the base 440 and the valve 42.

One of the cover 444 and the intermediate component 442 may include one or more fluid stops 464 that define one or more fluid openings 466. When the actuator 446 is in a first position (e.g., a fluid flow position), the windows 454 of the actuator 446 may be entirely or at least partially aligned with the one or more fluid openings 466 and pressurized liquid may flow from the fluid reservoir into the first chamber 456 and through the first port 410a and/or the second port 410b. When the actuator 446 is in a second position (e.g., a fluid blocked position), the windows 454 of the actuator 446 may be entirely or at least partially blocked by the fluid stops such that no flow of liquid passes from the fluid reservoir to the first chamber 456.

The intermediate component 442 may be configured to engage an outer surface of the fluid reservoir at or proximate an opening in the fluid reservoir. In one example, a bottom outer surface (not shown in FIG. 14) may be configured to engage an outer surface of a wall of the fluid reservoir to seal an opening in the fluid reservoir through which the manifold 400 may extend.

The base 440 may include an upper surface 468 that is configured to engage an interior surface of the fluid reservoir proximate the opening through which the manifold 400 may extend. In one example, the side wall of the fluid reservoir that defines the opening through which the manifold 400 may extend may be sandwich between the base 440 and the intermediate component 442. In such cases, the base 440 and the intermediate component may be coupled to each other and/or the inner and outer surface, respectively, of a wall of the fluid reservoir with adhesive, silicon adhesive, heat bonding, and/or other suitable hermetic coupling techniques. In some cases, silicon and/or other suitable resilient material may be positioned between two or more of the base 440, the intermediate component 442, and/or the wall of the fluid reservoir between the base 440.

The base 440 may include a recessed portion 470 with a plurality of openings 472 that may extend through (e.g., entirely through) the base 440. Although the plurality of openings 472 are depicted in the recessed portion 470, the recessed portion 470 may be omitted and the openings 470 may extend through a non-recessed portion of the base 440.

In operation, gas entering the manifold 400 may be pass from the second chamber 458 (e.g., openings in the intermediate component 442 at the second chamber 458) to the openings 472 and into the fluid reservoir. In some cases, the manifold 400 may include the valve 412 that may be configured to cover and seal the opening 472 until the pressure in the second chamber 458 reach or exceed a threshold pressure.

The valve 412 may be any suitable one-way valve as discussed herein or otherwise. In one example, the valve 412 may be coupled to the base 440 via the actuator 446 and the clip connector 448 and may be sized to cover the openings 472. As such, when the valve 412 is in a first position (e.g., a resting position), the valve 412 may cover the openings 472 and once a pressure in the openings 472 and/or the second chamber 458 reaches or exceeds a threshold pressure, the valve 412 may open to allow fluid to pass through the openings 472 until the pressure in the second chamber 458 and/or the openings 472 no longer reaches or exceeds the threshold pressure.

The valve 412 depicted in FIG. 14 may be formed from any suitable material. In one example, the valve 412 may be made from silicone, elastomers, thermoplastic elastomers (TPE), rubberized material, and/or other suitable materials.

FIG. 15 depicts a schematic view of the illustrative manifold 400 depicted in FIG. 13, where the manifold 400 is coupled to a wall of the water reservoir 270, 305. As depicted in FIG. 15, the manifold 400 may include the first port 410a configured to be coupled with CO₂ tubing, the second port 410b configured to be coupled with lens wash tubing and/or air supply tubing, and the third port 410c configured for coupling with irrigation tubing. Other suitable tubing may be coupled with the manifold 400, as desired. Further, as depicted in FIG. 14, the intermediate component 442, the cover 444, and the actuator 446 may form the second portion 400b of the manifold 400.

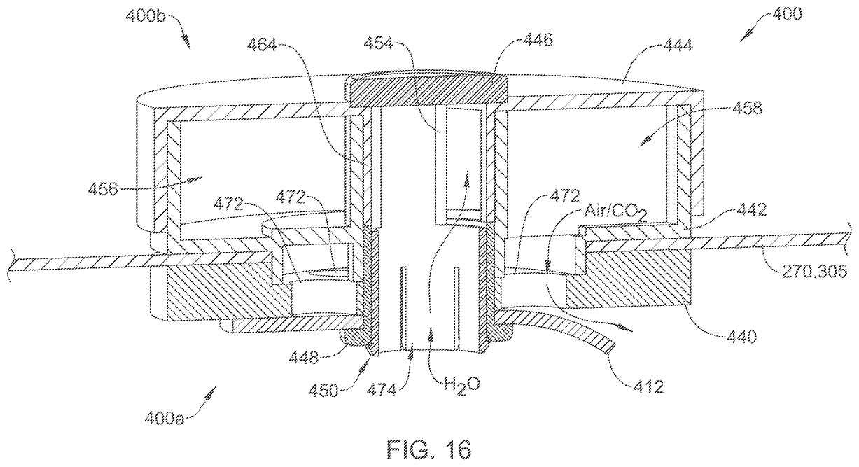

FIG. 16 depicts a central cross-sectional view of the manifold 400 coupled to a wall of the water reservoir 270, 305, with the actuator in an opened position. When gas enters the water reservoir 270, 305 from the second chamber 458 through the openings 472 and the opened valve 412, fluid may pass from the water reservoir 270, 305 through a central opening 474 of the actuator 446, through the windows 454 of the actuator 446 and into the first chamber 456.

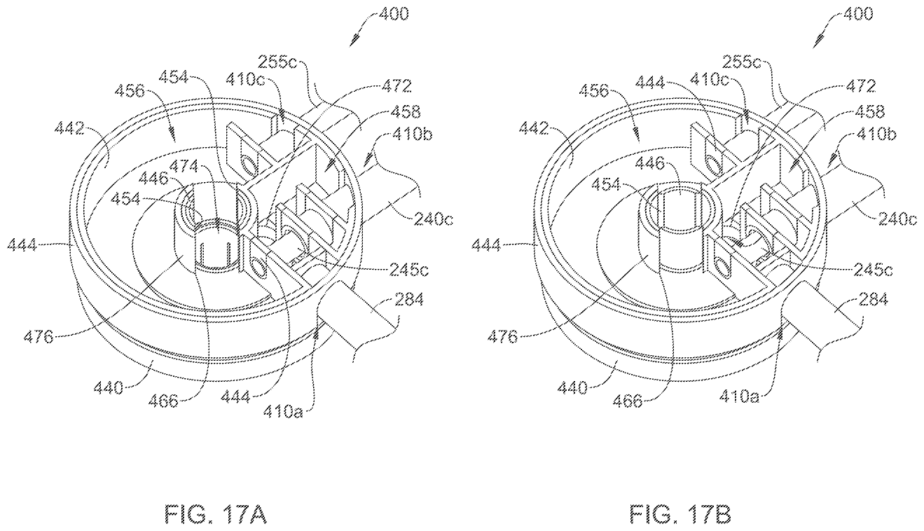

FIGS. 17A and 17B depict the manifold 400 coupled to tubing, where the top surface of the cover 444 of the manifold 400 has been removed such that first chamber 456 and the second chamber 458 can be viewed. As depicted in FIGS. 17A and 17B, the tubing has been secured in ports of the manifold, where the ports are at least partially formed by the intermediate component 442 and at least partially formed by the cover 444.

FIG. 17A depicts the actuator 446 in a first or actuated or opened position in which fluid from the fluid reservoir may flow into the manifold 400 when fluid is sufficiently pressurized within the fluid reservoir. In one example, pressurized fluid may flow from the fluid reservoir, into a central opening 474 of the actuator 446, through the windows 454 of the actuator 446, and through fluid openings 466 of a post 476 of the intermediate component 442 into the first chamber 456 and into one or both of the irrigation tubing 255s and the lens wash tubing 245c.

To pressurize the fluid in the fluid reservoir, gas or other suitable fluid may be passed through the air supply tubing 240c and/or the CO₂ tubing 245c into the second chamber 458, into the openings 472 of the base 440, which are open to the second chamber 458. Once pressure in the second chamber 458 and the openings 472 reaches or goes beyond a threshold value, the valve 412 (not depicted in FIGS. 17A and 17B) covering the openings 272 may open to allow the gas in the second chamber 458 to exit the manifold 400 and enter the fluid reservoir. Once the pressure in the second chamber 458 drops below the threshold value, the valve 412 may close (e.g., automatically) to prevent liquid from entering the openings 472 and the second chamber 458.

FIG. 17B depicts the actuator 446 in a second or resting or closed position in which the fluid from the fluid reservoir may be prevented from flowing into the first chamber 456 of the manifold 400 regardless of whether the pressure within the fluid reservoir. As depicted in FIG. 17B, the actuator 446 has been rotated relative to what is depicted in FIG. 17A, such that the actuator 446 covers the opening 466 in the post 476 to prevent any flow from the fluid reservoir to the first chamber 456. Utilizing an actuator in this manner may facilitate switch tubing connected to the manifold 400 and/or disconnecting the tubing from the endoscope umbilical without having to depressurize the fluid reservoir to prevent or mitigate leakage from the disconnected tubing or manifold 400.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A manifold configured to couple to a fluid source and a tubing assembly in fluid communication with an endoscope, the manifold comprising:
a first portion configured to be internal to the fluid source, the first portion comprising:
a first opening in fluid communication with a first through hole; and
a second opening in fluid communication with a second through hole; and
a second portion configured to be external to the fluid source, the second portion comprising:
a first port in fluid communication with the first through hole, the first port is configured to couple with a first tubing configured to be in fluid communication with the endoscope; and
a second port in fluid communication with the second through hole, the second port is configured to couple to a second tubing to be in fluid communication with the endoscope.

2. The manifold of claim 1, further comprising:
a third opening in the first portion, the third opening is in fluid communication with a third through hole and the first port; and
a valve in fluid communication with the third opening, wherein when pressure in the third through hole reaches or goes beyond a threshold, the valve allows gas to output from the third opening, and when pressure in the third through hole has not reached the threshold, the valve prevents gas from exiting through the third opening.

3. The manifold of claim 1, wherein:
the first portion comprises a third opening in fluid communication with a third through hole and the first port;
the second portion comprises a third port in fluid communication with the third through hole and the third opening; and
the third opening is configured to receive gas passing through one or both of the first port and the third port.

4. The manifold of claim 1, further comprising:
an interface configured to engage a wall of the fluid source and seal an opening in the wall of the fluid source in which one or both of the first portion and the second portion extends.

5. The manifold of claim 4, wherein the interface defines a perimeter having a portion that is configured to be parallel to a bottom surface of the fluid source.

6. The manifold of claim 1, further comprising:
a connector configured to connect the first portion to the second portion.

7. The manifold of claim 1, wherein connector comprises:
a first set of threads extending around the first portion;
a second set of threads extending around the second portion; and
the first set of threads and the second set of threads are configured to threadedly engage one another to secure the first portion and the second portion to the fluid source.

8. The manifold of claim 1, further comprising:
an actuator configured to be adjusted to adjust flow of fluid through the second portion.

9. The manifold of claim 8, wherein:
when the actuator is in a first position, liquid in the fluid source is allowed to flow from the fluid source through the first port; and
when the actuator is in a second position, liquid in the fluid source is prevented from flowing from the fluid source through the first port.

10. The manifold of claim 8, wherein the actuator is configured to couple the first portion with the second portion.

11. A fluid reservoir assembly configured to couple to a tubing assembly in fluid communication with an endoscope, the fluid reservoir assembly comprising:
a container having a first opening and a second opening;
a cap configured to couple to the container and cover the first opening; and
a manifold configured to couple to the container and cover the second opening, the manifold having a first port and a second port positioned exterior of the container when the manifold is coupled to the container; and
wherein the first port and the second port are in fluid communication with an interior of the container.

12. The fluid reservoir assembly of claim 11, wherein the manifold further comprises:
a third port, wherein when the manifold is coupled to the container, third port is positioned exterior of the container and is in fluid communication with the interior of the container.

13. The fluid reservoir assembly of claim 11, wherein:
the container has a first end, a second end, and a side wall extending between the first end and the second end; and
the first opening extends through the first end and the second opening extends through the side wall proximate the second end.

14. The fluid reservoir assembly of claim 13, wherein the manifold comprises a perimeter, a portion of which is parallel to the second end of the container.

15. The fluid reservoir assembly of claim 11, wherein:
the manifold comprises a first portion configured to be positioned exterior of the container and a second portion configured to be positioned in the interior of the container; and
the first portion and the second portion are configured to couple to one another and fluidly seal the first opening.

16. A medical device assembly comprising:
an endoscope;
a tubing assembly configured to couple to the endoscope, the tubing assembly having a first tubing;
a fluid reservoir; and
a manifold, the manifold is configured to couple to the fluid reservoir, the manifold includes:
a first port configured to be in fluid communication with liquid in the fluid reservoir and the first tubing of the tubing assembly; and
a second port configured to be in fluid communication with a pressurized gas, an interior of the fluid reservoir, and the first tubing of the tubing assembly.

17. The assembly of claim 16, wherein the manifold further comprises:
a third port configured to be in fluid communication with the liquid in the fluid reservoir and a second tubing of the tubing assembly.

18. The assembly of claim 16, wherein:
the fluid reservoir has a first end, a second end, a side wall extending between the first end and the second end, and an opening extending through the side wall; and
the manifold is configured to extend through the opening in the side wall.

19. The assembly of claim 18, wherein:
the manifold comprises a first portion configured to be positioned exterior of the fluid reservoir and a second portion configured to be positioned in the interior of the fluid reservoir; and
the first portion and the second portion are configured to couple to one another and fluidly seal the opening.

20. The assembly of claim 16, wherein the manifold comprises a one-way valve configured to be positioned interior of the fluid reservoir.
